(19) United States
(12) Patent Application Publication
(10) Pub. No.: US 2018/0333141 A1
(43) Pub. Date: Nov. 22, 2018

(54) NEUROSURGICAL MRI-GUIDED ULTRASOUND VIA MULTI-MODAL IMAGE REGISTRATION AND MULTI-SENSOR FUSION

(71) Applicants: Utsav PARDASANI, Toronto (CA); Ali KHAN, Toronto (CA)

(72) Inventors: Utsav PARDASANI, Toronto (CA); Ali KHAN, Toronto (CA)

(21) Appl. No.: 15/777,263

(22) PCT Filed: Nov. 19, 2015
(86) PCT No.: PCT/IB2015/058984

§ 371 Date: May 18, 2018

(51) Int. Cl. A61B 8/08; A61B 8/00; A61B 10/02; A61B 34/20

(52) U.S. Cl. CPC A61B 8/5261; A61B 8/4254; A61B 8/483; A61B 8/58; A61N 1/0534; A61B 8/0841; A61B 8/0808; A61B 34/20; A61B 2562/0219; A61B 10/0233

(57) ABSTRACT

Ultrasound's value in the neurosurgical operating room is maximized when fused with pre-operative images, The disclosed system enables real-time multimodal image fusion by estimating the ultrasound's pose with use of an image-based registration constrained by sensor measurements and pre-operative image data. Once the ultrasound data is collected and viewed, it can be used to update the pre-operative image, and make changes to the pre-operative plan. If a surgical navigation system is available for integration, the system has the capacity to produce a 3D ultrasound volume, probe-to-tracker calibration, as well as an optical-to-patient registration. This 3D ultrasound volume, and optical-to-patient registration can be updated with conventional deformable registration algorithms and tracked ultrasound data from the surgical navigation system. The system can also enable real-time image-guidance of tools visible under ultrasound by providing context from the registered pre-operative image when said tools are instrumented with sensors to help constrain their pose.

### TECHNICAL FIELD

The present disclosure is generally related to neurosurgical or medical procedures, and more specifically the viewing of a volumetric three dimensional (3D) image reformatted to match the pose of an intraoperative imaging probe.

### BACKGROUND

In the field of medicine, imaging and image guidance are a significant component of clinical care. From diagnosis and monitoring of disease, to planning of the surgical approach, to guidance during procedures and follow-up after the procedure is complete, imaging and image guidance provides effective and multifaceted treatment approaches, for a variety of procedures, including surgery and radiation therapy. Targeted stem cell delivery, adaptive chemotherapy regimes, and radiation therapy are only a few examples of procedures utilizing imaging guidance in the medical field.

Advanced imaging modalities such as Magnetic Resonance Imaging (“MRI”) have led to improved rates and accuracy of detection, diagnosis and staging in several fields of medicine including neurology, where imaging of diseases such as brain cancer, stroke, Intra-Cerebral Hemorrhage (“ICH”), and neurodegenerative diseases, such as Parkinson's and Alzheimer's, are performed. As an imaging modality, MRI enables three-dimensional visualization of tissue with high contrast in soft tissue without the use of ionizing radiation. This modality is often used in conjunction with other modalities such as Ultrasound (“US”), Positron Emission Tomography (“PET”) and Computed X-ray Tomography (“CT”), by examining the same tissue using the different physical principals available with each modality. CT is often used to visualize boney structures, and blood vessels when used in conjunction with an intra-venous agent such as an iodinated contrast agent. MRI may also be performed using a similar contrast agent, such as an intra-venous gadolinium based contrast agent which has pharmaco-kinetic properties that enable visualization of tumors, and break-down of the blood brain barrier. These multi-modality solutions can provide varying degrees of contrast between different tissue types, tissue function, and disease states. Imaging modalities can be used in isolation, or in combination to better differentiate and diagnose disease.

In neurosurgery, for example, brain tumors are typically excised through an open craniotomy approach guided by imaging. The data collected in these solutions typically consists of CT scans with an associated contrast agent, such as iodinated contrast agent, as well as MRI scans with an associated contrast agent, such as gadolinium contrast agent. Also, optical imaging is often used in the form of a microscope to differentiate the boundaries of the tumor from healthy tissue, known as the peripheral zone. Tracking of instruments relative to the patient and the associated imaging data is also often achieved by way of external hardware systems such as mechanical arms, or radiofrequency or optical tracking devices. As a set, these devices are commonly referred to as surgical navigation systems.

These surgical navigation systems may include the capacity to track an ultrasound probe or another intra-operative imaging modality in order to correct anatomical changes since the intra-operative image was made, to provide enhanced visualization of the tumour or target, and/or to register the surgical navigation system's tracking system to the patient. Herein, this class of systems shall be referred to as intraoperative multi-modality imaging systems.

Conventional intraoperative multi-modality imaging systems that are attached to state-of-the-art neuronavigation systems bring additional hardware, set-up time, and complexity to a procedure. This is especially the case if a neurosurgeon only wants a confirmation operation plan prior to opening the dura. Thus, there is a need to simplify conventional tracked ultrasound neuronavigation systems so that they can offer a quick check using intra-operative ultrasound prior to opening the dura in surgery with or without neuronavigation guidance.

### SUMMARY

Ultrasound's value in the neurosurgical operating room is maximized when fused with pre-operative images. The disclosed system enables real-time multi-modality image fusion by estimating the ultrasound's pose with use of an image-based registration constrained by sensor measurements, and pre-operative image data. The system enables multi-modality image fusion independent of whether a surgeon wishes to continue the procedure using a conventional surgical navigation system, a stereotaxic frame, or using ultrasound guidance. Once the ultrasound data is collected and viewed, it can be used to update the pre-operative image, and make changes to the pre-operative plan. If a surgical navigation system is available for integration, prior to the dural opening, the system has the capacity to produce a 3D ultrasound volume, probe-to-tracker calibration, as well as an optical-to-patient registration. This 3D ultrasound volume, and optical-to-patient registration can be updated with conventional deformable registration algorithms and tracked ultrasound data from the surgical navigation system. The system can also enable real-time image-guidance of tools visible under ultrasound by providing context from the registered pre-operative image.

Once a neurosurgeon has confirmed the operation plan under ultrasound with the dura intact, the disclosed system provides the option of supporting ultrasound-guidance of procedures (such as Deep Brain Stimulation (DBS) Probe placement, Tumour Biopsy, or port cannulation) with or without the use of a surgical navigation system.

The disclosed system would enhance procedures that do not make use of a surgical navigation system. (Such as those employing stereotaxic frames). The disclosed system can also enable the multi-modal neuroimaging of neonatal brains through the fontanelle without the burden and expense of a surgical navigation system.

In emergency situations where an expensive modality such as MRI is unavailable, the disclosed system can enable the augmentation of a less expensive modality such as CT with Ultrasound to better inform a procedure.

### BRIEF DESCRIPTION OF THE DRAWINGS

Embodiments will now be described, by way of example only, with reference to the drawings, in which:

FIG. 1A illustrates the craniotomy site with the dura intact through which the ultrasound probe will image the patient.

FIG. 1B shows some components of an exemplary system displaying co-registered ultrasound and MRI images.

FIG. 1C shows another exemplary system enhanced to include tracking of a surgical tool by combining image-based tracking of the tool and sensor readings from a variety of sources.

FIG. 1D shows another exemplary system that employs readings from a variety of sensors, as well as a conventional neurosurgical navigation system with optical tracking sensors.

FIG. 2A is a flow chart illustrating a workflow involved in a surgical procedure using the disclosed system.

FIG. 2B is a flow chart illustrating aspects of the novel method for estimating a US probe pose for the systems shown in FIGS. 1A-1D, a subset of block 204 in FIG. 2A.

FIG. 2C is a flow chart illustrating a workflow in which the described system can benefit the workflow when used with a conventional neurosurgical guidance system that employs an optical or magnetic tracking system to track a US probe.

### DETAILED DESCRIPTION

Various embodiments and aspects of the disclosure will be described with reference to details discussed below. The following description and drawings are illustrative of the disclosure and are not to be construed as limiting the disclosure. Numerous specific details are described to provide a thorough understanding of various embodiments of the present disclosure. However, in certain instances, well-known or conventional details are not described in order to provide a concise discussion of embodiments of the present disclosure.

As used herein, the terms “comprises” and “comprising” are to be construed as being inclusive and open ended, and not exclusive. Specifically, when used in the specification and claims, the terms “comprises” and “comprising” and variations thereof mean the specified features, steps or components are included. These terms are not to be interpreted to exclude the presence of other features, steps or components.

As used herein, the term “exemplary” means “serving as an example, instance, or illustration,” and should not be construed as preferred or advantageous over other configurations disclosed herein.

As used herein, the terms “about”, “approximately”, and “substantially” are meant to cover variations that may exist in the upper and lower limits of the ranges of values, such as variations in properties, parameters, and dimensions. In one non-limiting example, the terms “about”, “approximately”, and “substantially” mean plus or minus 10 percent or less

Unless defined otherwise, all technical and scientific terms used herein are intended to have the same meaning as commonly understood by one of ordinary skill in the art. Unless otherwise indicated, such as through context, as used herein, the following terms are intended to have the following meanings:

As used herein the phrase “intraoperative” refers to an action, process, method, event or step that occurs or is carried out during at least a portion of a medical procedure. Intraoperative, as defined herein, is not limited to surgical procedures, and may refer to other types of medical procedures, such as diagnostic and therapeutic procedures.

Embodiments of the present disclosure provide imaging devices that are insertable into a subject or patient for imaging internal tissues, and methods of use thereof. Some embodiments of the present disclosure relate to minimally invasive medical procedures that are performed via an access port, whereby surgery, diagnostic imaging, therapy, or other medical procedures (e.g. minimally invasive medical procedures) are performed based on access to internal tissue through the access port.

The present disclosure is generally related to medical procedures, neurosurgery.

In the example of a port-based surgery, a surgeon or robotic surgical system may perform a surgical procedure involving tumor resection in which the residual tumor remaining after is minimized, while also minimizing the trauma to the healthy white and grey matter of the brain. In such procedures, trauma may occur, for example, due to contact with the access port, stress to the brain matter, unintentional impact with surgical devices, and/or accidental resection of healthy tissue. A key to minimizing trauma is ensuring that the spatial location of the patient as understood by the surgeon and the surgical system is as accurate as possible.

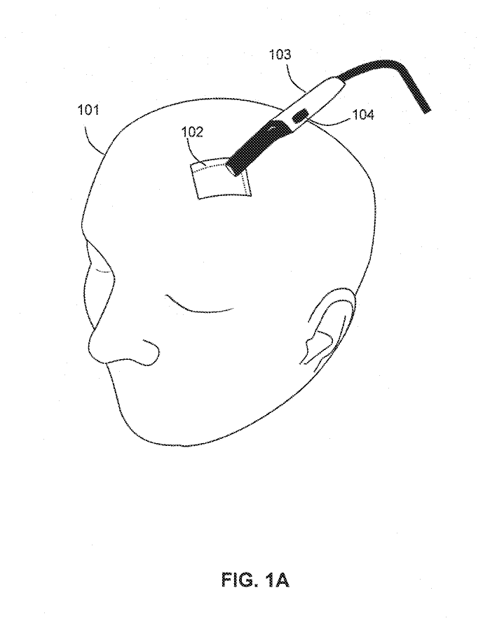

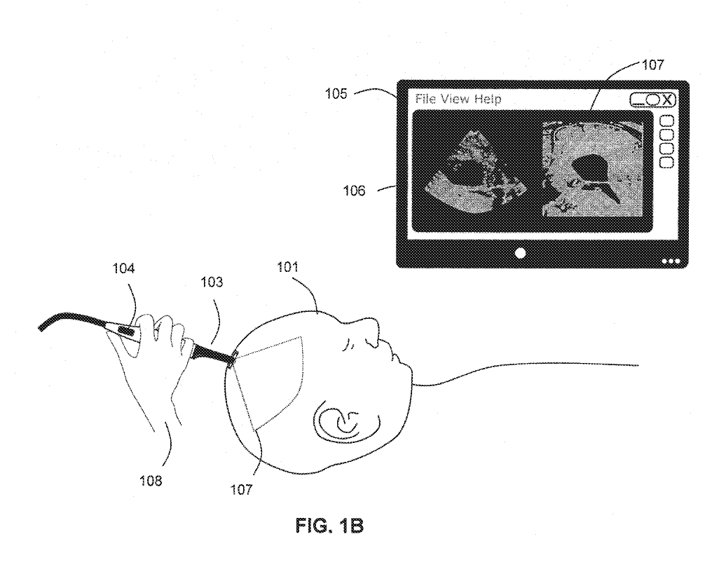

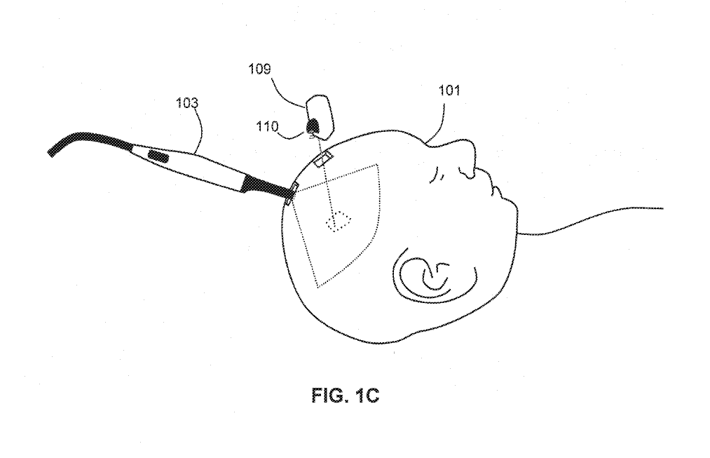

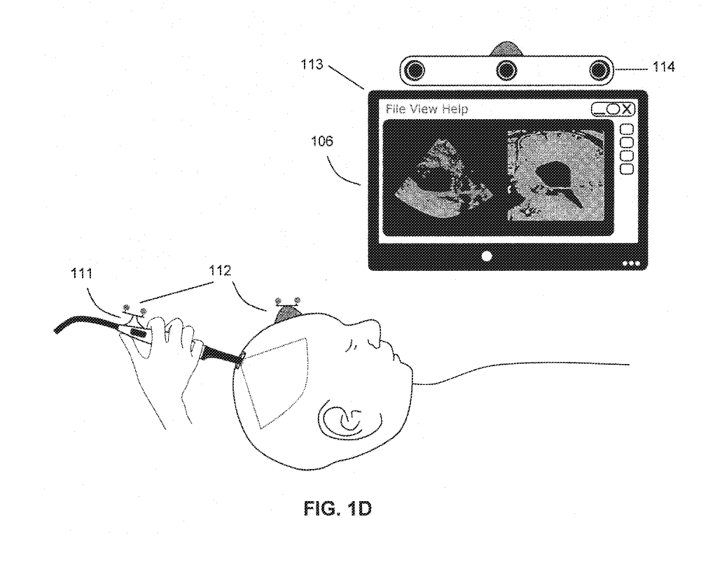

FIG. 1A illustrates the craniotomy site with the dura intact through which the ultrasound probe will image the patient. FIG. 1A illustrates the use of an US probe 103 held by the surgeon instrumented with a sensor 104 to image through a given craniotomy site 102 of patient 101. In FIG. 1B, the pre-operative image 107 is shown reformatted to match the intra-operative ultrasound image 106 on display 105 as the surgeon 108 moves the probe.

In the example shown in FIGS. 1A, 1B, 1C, and 1D, the US probe 103 may have the sensor(s) 104 built-in, or attached externally temporarily or permanently using a fixation mechanism. The sensor(s) may be wireless or wired. In the examples shown in FIGS. 1A, 1B, and 1C, and 1D, the US probe 103 may be any variety of US transducers including 3D probes, or burr-hole transducers.

Sensor 104 in FIG. 1A can be any combination of sensors that can help constrain the registration of the ultrasound image to the MRI volume. FIG. 1B shows some components of an exemplary system displaying co-registered ultrasound and MRI images, As shown in FIG. 1B, sensor 104 is an inertial measurement unit, however the probe 103 can be also instrumented with time-of-flight range finders, ultrasonic range finders, magnetometers, strain sensors, mechanical linkages, magnetic tracking systems or optical tracking systems.

An intra-operative multi-modal display system 105 comprising a computer, display, input devices, and acquisition hardware, shows reformatted volumetric pre-operative images and/or US probe placement guidance annotations to surgeon 108 during his procedure.

The present application includes the possibility of incorporating image-based tracking of tools 109 under ultrasound guidance through one or more craniotomy sites. FIG. 1C shows another exemplary system enhanced to include tracking of a surgical tool by combining image-based tracking of the tool and sensor readings from a variety of sources. The tool's pose, similar to the ultrasound probe's pose can be constrained using any combination of sensors 110 and its location in the US image. In this exemplary embodiment, the orientation of the tool is constrained with an IMU, and the depth is constrained with an optical time-of-flight sensor. Thus, only a cross-section of the tool is needed under US viewing in order to fully constrain its pose.

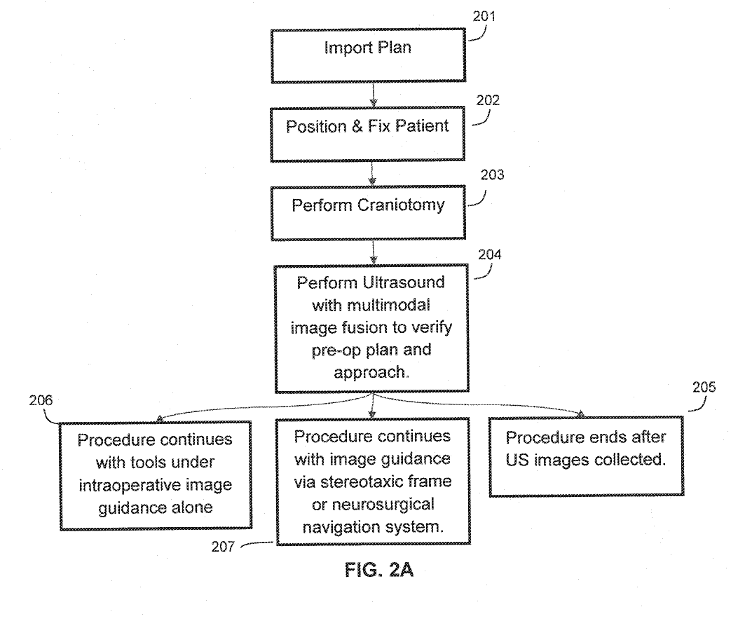

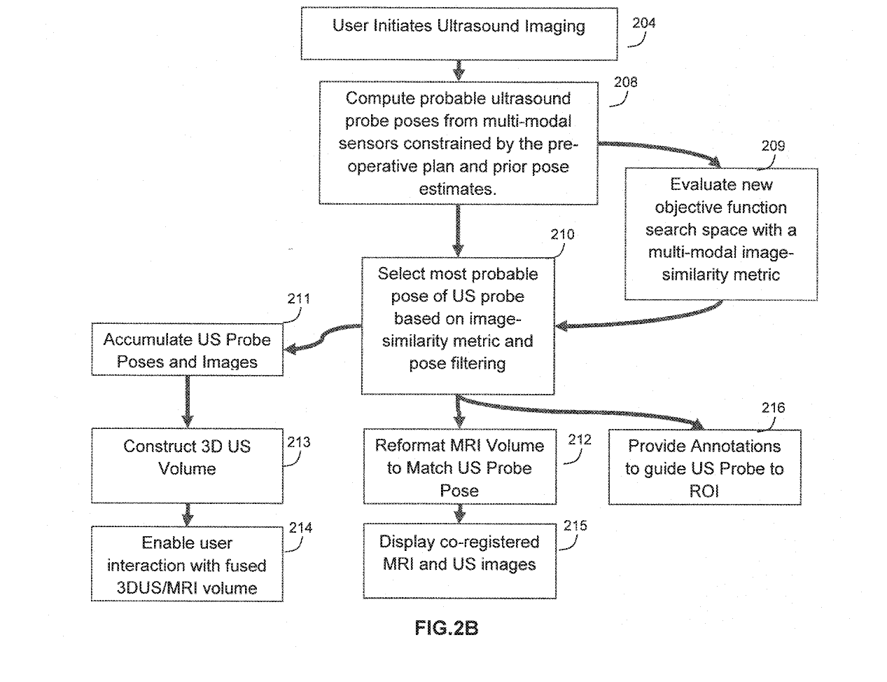

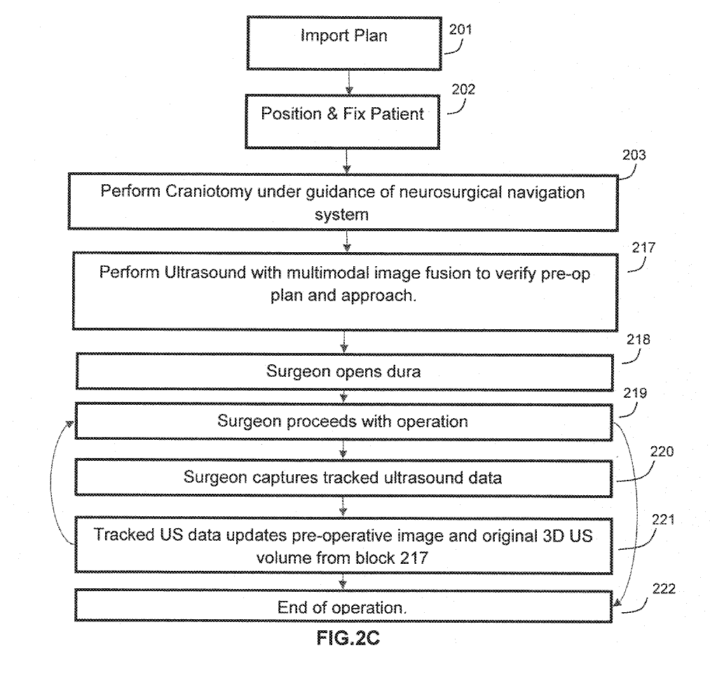

FIG. 2A is a flow chart illustrating a workflow involved in a surgical procedure using the disclosed system. At the onset of FIG. 2A, the port-based surgical plan is imported (Block 201). A detailed description of the process to create and select a surgical plan is outlined in international publication WO/2014/139024, entitled “PLANNING, NAVIGATION AND SIMULATION SYSTEMS AND METHODS FOR MINIMALLY INVASIVE THERAPY”, which claims priority to United States Provisional Patent Application Serial Nos. 61/800,155 and 61/924,993, which are all hereby incorporated by reference in their entirety.

Once the plan has been imported into the navigation system (Block 201), the patient is placed on a surgical bed. The head position can be placed using any means available to the surgeon (Block 202). The surgeon will then perform a craniotomy using any means available to the surgeon. (Block 203). As an example, this may be accomplished by using a neurosurgical navigation system, a stereotaxic frame, or using fiducials.

Next, prior to opening the dura of the patient, the surgeon performs an ultrasound session using the US probe instrumented with a sensor (Block 204). In the exemplary system shown in FIGS. 1A, 1B, and 1C this sensor is an inertial measurement unit (Block 104). As seen in FIG. 2A, once the multi-modal session is over, the dura may be opened and the procedure can continue under US guidance (Block 206), under pre-operative image-guidance (Block 207), or the procedure can be ended based on the information collected (Block 205).

Referring now to FIG, 2B, a flow chart is shown illustrating a method involved in registration block 204 as outlined in FIG. 2A, in greater detail. Referring to FIG. 2B, an ultrasound session is initiated (Block 204).

The next step is to compute probable ultrasound probe poses from multi-modal sensors constrained by the pre-operative plan and prior pose estimates (Block 208). A further step of evaluating new objective function search space with a multi-modal image-similarity metric (Block 209) may be initiated, or the process may advance directly to the next step of selecting most probable pose of US probe based on image-similarity metric and pose filtering (Block 210).

A variety of optimizers may be used to find the most likely pose of the US probe (Block 210). These include optimizers that calculate the local derivative of the objective function to find a global optima. Also in this step (Block 210) filtering sensor estimates is used generate an objective function search space and to bias the registration metric against false local minima. This filtering may include any number of algorithms for generating pose estimates including Kalman Filtering, Extended Kalman Filtering, Unscented Kalman Filtering, and Particle/Swarm filtering.

After a pose is selected (Block 210), the system's algorithm for constraining a US-pose can be utilized in a variety of beneficial ways by the surgeon, which is represented by three paths in FIG. 2B. The first path is to accumulate the US probe poses and images (Block 211) where 3D US volumes can be created (Block 213) and visualized by the surgeon in conjunction with pre-operative images (Block 214). An example of pre-operative images may include pre-operative MRI volumes.

Alternatively, the surgeon's intraoperative imaging may be guided by pre-operative images displayed on the screen that are processed and reformatted in real-time (Block 212) or using display annotations instructing the surgeon which direction to move the US probe (Block 216).

In a second path, a live view of the MR image volume can be created and reformatted to match the US probe (Block 212). The display of co-registered pre-operative and US images (Block 215) is then presented to the surgeon (or user) to aid in the understanding of the surgical site.

Alternatively in a third path (from Block 210), a further step of provide annotations to guide US Probe to region of interest (ROI) (Block 216) can be established. By selecting ROIs in the pre-operative volume (Block 216), a surgeon can receive guidance from the system on where to place the US probe to find a given region in US.

Tracked data from a conventional neurosurgical tracking system can be fused with the US pose estimates produced by the disclosed system to produce a patient to pre-operative image volume registration, as well as a tracking tool to US probe calibration Such a system is depicted in FIG. 1D and captured in the workflow shown in FIG. 2C.

This invention also includes the possibility of integrating a conventional surgical navigation system. FIG. 1D shows another exemplary system that employs readings from a variety of sensors, as well as a conventional neurosurgical navigation system with optical tracking sensors. As shown in FIG. 1D, a probe tracking tool 111 may be tracked with a tracking reference 112 on the tool and/or a tracking reference 112 on the patient. The tracking reference 112 relays the data to neurosurgical navigation system 113 which utilizes optical tracking sensors 114 to receive data from tracking reference 112 and outputs the information onto display 106.

As seen in FIG. 1D, the disclosed invention would enable US guidance to continue if line-of-sight is lost on the tracking reference 112 or the probe tracking tool 111. In this embodiment the disclosed invention would also enable calibration of the US probe face to the tracking system in real-time, as well as an automatic registration. Once the dura is opened, tracked US data can be used to update the previously acquired 3D US volume and pre-operative image with a deformable registration algorithm.

Further, FIG. 2C is a flow chart that illustrates this workflow in which the described system can benefit the workflow when used with a conventional neurosurgical guidance system as seen in FIG. 1D that employs an optical or magnetic tracking system to track a US probe. The first step of FIG. 2C is to import a plan (Block 201).

Once the plan has been imported into the navigation system (Block 201), the patient is placed on a surgical bed. The head position can be placed using any means available to the surgeon (Block 202). The surgeon will then perform a craniotomy using any means available to the surgeon. (Block 203).

The next step is to perform ultrasound registration with multimodal image fusion to verify pre-operative plan and approach (Block 217). The result is to produce probe calibration data, optical-patient registration data and/or 3D US volume data.

The surgeon will then open the patient's dura (Block 218) and then continues on with the operation (Block 219). If all goes, the surgeon may jump to the last step of ending the operation (Block 222).

Alternatively, the surgeon may proceed with the operation to the next step of capturing tracked ultrasound data (Block 220). Thereafter, the tracked US data updates the pre-operative image and original 3D US volume (Block 221) captured previously (from Block 217).

At this point, the surgeon may jump to the last step of ending the operation (Block 222) or proceed further on with the operation (Block 219).

Furthermore, in the exemplary embodiment including integration with a conventional surgical navigation system, any number of sensors, such as inertial measurement units can be attached to the tracking system, or patient reference to aid in the constraining of the US probe's registration if line-of-sight is interrupted.

A key aspect of the invention is the ability to display guidance to the surgeon as to how to place the ultrasound probe to reach an ROI, as well as aiding the interpretation of the ultrasound images with the pre-operative volume.

The disclosed invention also includes the embodiment where the reformatted MRI volume is processed to show the user the zone of positioning uncertainty with the ultrasound image.

The disclosed invention includes the capacity to process the pre-operative volume into thicker slices parallel to the US probe imaging plane to reflect higher out-of-imaging-plane pose inaccuracy in the ultrasound probe pose estimates.

The disclosed invention includes the embodiment where the pre-operative volume is processed to include neighboring data with consideration for the variability in US slice thickness throughout its imaging plane based on focal depth(s).

The disclosed invention includes the embodiment where the quality of the intra-operative modality's images is processed to inform the reconstruction of 3D Ultrasound volumes, image registration and US probe pose calculation which can be seen in Blocks 208-211 of FIG. 2B. An example of this is de-weighting ultrasound slices that have poor coupling.

A further aspect of this invention, as described in FIG. 2B, is the capacity of the system to produce a real-time ultrasound pose estimate from a single US slice by constraining the search space of a multi-modal image registration algorithm to a geometry defined by the pre-operative plan, volumetric data from the pre-operative image, and sensor readings that help constrain the pose of the US probe. The constrained region that the image-registration algorithm acts within as the objective function search space with a multi-modal similarity metric being the objective function.

A further aspect of this invention is that the geometric constraints on the objective function search-space can be derived from segmentations of the pre-operative image data. The exemplary embodiment incorporates the segmentation of the dura mater to constrain the search space.

A further aspect of this invention is that the geometric constraint of the objective function search space can be enhanced with sensor readings from external tools such as 3D scanners, or photographs and video from single or multiple sources made with or without cameras that have attached sensors, (such as the IMU on a tablet).

According to one aspect of the present application, one purpose of the multi-modal imaging system, is to provide tools to the neurosurgeon that will lead to the most informed, least damaging neurosurgical operations. In addition to removal of brain tumors and intracranial hemorrhages (ICH), the multi-modal imaging system can also be applied to a brain biopsy, a functional/deep-brain stimulation, a catheter/shunt placement procedure, open craniotomies, endonasal/skull-based/ENT, spine procedures, and other parts of the body such as breast biopsies, liver biopsies, etc. While several examples have been provided, aspects of the present disclosure may be applied to any suitable medical procedure.

Those skilled in the relevant arts will appreciate that there are numerous segmentation techniques available and one or more of the techniques may be applied to the present example. Non-limiting examples include atlas-based methods, intensity based methods, and shape based-methods.

Those skilled in the relevant arts will appreciate that there are numerous registration techniques available and one or more of the techniques may be applied to the present example. Non-limiting examples include intensity-based methods that compare intensity patterns in images via correlation metrics, while feature-based methods find correspondence between image features such as points, lines, and contours. Image registration methods may also be classified according to the transformation models they use to relate the target image space to the reference image space. Another classification can be made between single-modality and multi-modality methods. Single-modality methods typically register images in the same modality acquired by the same scanner or sensor type, for example, a series of magnetic resonance (MR) images may be co-registered, while multi-modality registration methods are used to register images acquired by different scanner or sensor types, for example in magnetic resonance imaging (MRI) and positron emission tomography (PET). In the present disclosure, multi-modality registration methods may be used in medical imaging of the head and/or brain as images of a subject are frequently obtained from different scanners. Examples include registration of brain computerized tomography (CT)/MRI images or PET/CT images for tumor localization, registration of contrast-enhanced CT images against non-contrast-enhanced CT images, and registration of ultrasound and CT to patient in physical space.

The specific embodiments described above have been shown by way of example, and it should be understood that these embodiments may be susceptible to various modifications and alternative forms, It should be further understood that the claims are not intended to be limited to the particular forms disclosed, but rather to cover modifications, equivalents, and alternatives falling within the spirit and scope of this disclosure.

1. A method of determining an ultrasound probe pose in three-dimensional space during a medical procedure, having the steps of:
a. receiving pre-operative image data;
b. receiving ultrasound image data using an ultrasound probe;
c. receiving sensor readings; and
d. applying an image-registration algorithm between the ultrasound image data and the pre-operative image data constrained by data from pre-operative images and sensor readings to a range of possible probe poses to create an estimate of the probe pose.

2. The method of claim 1, wherein the ultrasound image data is selected from a group consisting of three-dimensional data and two-dimensional data.

3. The system of claim 2, wherein said sensor is one or more sensors that constrains the pose of the ultrasound probe.

4. The system of claim 3, wherein said sensor is an inertial measurement unit sensor.

5. The method of claim 1, further comprising acquiring additional geometric constraints intraoperatively from a portable device having a camera and a built-in inertial measurement unit.

6. The method of claim 1, wherein the sensor is one of either a magnetic or optical tracking system such that registration is partially constrained with an estimate of patient initial orientation with respect to ground.

7. The method of claim 1, wherein registration is further constrained with three-dimensional surface information of cortex boundary.

8. The method of claim 7, wherein registration is further constrained using segmentation from said pre-operative images.

9. The method of claim 7, wherein the segmentation is further refined using a mathematical model of brain-shift deformation.

10. The method of claim 7, wherein registration is further constrained using surfaces created from stereoscopic images, structured light, or laser scanning.

11. The method of claim 1, wherein the pose estimate is further refined using a statistical method for estimating ultrasound movement from image data.

12. The method of claim 11 wherein the pose estimate is further refined using speckle-tracking.

13. The method of claim 1, further comprising refining a view of the ultrasound probe with said pre-operative image to account for brainshift.

14. The method of claim 1, further comprising processing a view of the ultrasound device with said pre-operative image to show a user the zone of positioning uncertainty with the ultrasound image.

15. The method of claim 1, wherein signals from at least one sensor are filtered for one of either determining a range of possible ultrasound poses or refining a pose estimate.

16. The system of claim 15, wherein the said signals is related to information selected from a group consisting of position information, velocity information, acceleration information, angular velocity information, angular acceleration information, and orientation information.

17. The method of claim 15, wherein said filtering is selected from a group consisting of Kalman filtering, extended Kalman filtering, unscented Kalman filtering, and Particle/Swarm filtering.

18. The method of claim 1, wherein the pre-operative image data is annotated with a pre-operative plan to constrain said image-registration algorithm.

19. A system for visualizing ultrasound images in three-dimensional space during a medical procedure, comprising:
a. an ultrasound probe;
b. at least one sensor for measuring pose information from said ultrasound probe; and
c. an intra-operative multi-modal display system for
i. receiving pre-operative image data and pre-operative plan data to estimate a range of possible poses;
ii. receiving ultrasound image data from said ultrasound probe;
iii. estimating pose of the ultrasound probe by executing an image-registration algorithm constrained to the estimated range of possible poses;
iv. receiving position data from the at least one sensor and in response refining the estimated pose of the ultrasound probe; and
v. displaying the pre-operative image data with information from the ultrasound image data.

20. The system of claim 19, wherein the sensor is selected from a group consisting of time-of-flight sensor, camera sensor, magnetometer, laser scanner, and ultrasonic sensor.

21. The system of claim 19, wherein said pose information is selected from a group consisting of position information, velocity information, acceleration information, angular velocity information, and orientation information.

22. The method of claim 1, wherein a surgical tool, visible in the ultrasound images, has its position estimated with the data in the ultrasound image, and additional sensors to help constrain the possible poses of the tool.

23. The method of claim 22, wherein said tool is selected from a group consisting of deep brain stimulator probe, ultrasonic aspirator, and biopsy needle.

24. The method of claim 22, wherein said tool is instrumented with a sensor selected from a group consisting of time-of-flight sensor, ultrasonic range finder, camera, magnetometer and inertial measurement unit.

25. The system of claim 19, for visualizing a surgical tool with its position estimated from the data in the ultrasound image and additional sensors.
